(19) United States
(12) Patent Application Publication
(10) Pub. No.: US 2011/0170060 A1
(43) Pub. Date: Jul. 14, 2011

(54) Gaze Tracking Using Polarized Light

(76) Inventor: Gary B. Gordon, Saratoga, CA (US)

(21) Appl. No.: 12/684,613

(22) Filed: Jan. 8, 2010

(51) Int. Cl. A61B 3/113; A61B 3/14

(52) U.S. Cl. 351/206; 351/246

(57) ABSTRACT

A gaze-tracking system uses separate “glint” and “pupil” images to determine the position of the pupil relative to the position of the glint. Since separate images are obtained, the exposures can be independently optimized for each image's intended purpose (e.g., locating the glint or locating the pupil, respectively). Polarizers are used to eliminate the glint in one image. This more saliently reveals the pupil, allowing its position relative to the glint to be determined more precisely, and enhancing the accuracy and robustness of the system.

### BACKGROUND

There are a number of eye-tracking techniques used in the prior art directed toward determining a viewer's gaze target position. In some cases, such techniques can permit persons to control some aspects of their environment using eye movement, as for example, enabling quadriplegics to control a computer or other device to read, to communicate, and to perform other useful tasks.

One class of gaze-tracking techniques uses illumination to produce a “glint” on an eye. The direction the viewer is looking is then determined from the position of the pupil relative to the position of the glint in an image of the eye. Such devices have been manufactured for decades and represent a great benefit to their users. Nonetheless they variously suffer from imperfect accuracy, restrictive lighting requirements, and not working at all with some individuals. The problem has always been the inordinate degree of finesse required to measure the relative positions of the pupil and the glint. More specifically, the problem has been how to accurately locate the centroid of a pupil when it is partially obscured by a glint.

### BRIEF DESCRIPTION OF THE DRAWINGS

FIG. 1 is a perspective and partially exploded view of a viewer and a gaze-tracking system in accordance with the present invention.

FIG. 2 is a region of a “glint” image obtainable using the system of FIG. 1.

FIG. 3 is a region of a “pupil” image obtainable using the system of FIG. 1.

FIG. 4 is a schematic diagram of the gaze-tracking system of FIG. 1.

FIG. 5 is a flow chart of a gaze-tracking process in accordance with the invention and implemented in the system of FIG. 1.

### DETAILED DESCRIPTION

In accordance with the present invention, distinct and separate “glint” and “pupil” images are obtained. For the pupil image, polarizing filters are used to remove the reflected glint, leaving scattered light to reveal the iris and pupil. Since the glint is a reflection, the polarizing filters are not used to attenuate reflected light in the glint image. Also, since separate glint and pupil images are obtained, different exposures (time and intensity) can be selected to optimize the detectability of the main subject (glint or pupil) of each image.

Polarized light has a history of countless uses, and indeed in photography a polarizer is one of the most commonly used filters. Polarized illumination and sensing is especially applicable for photographing shiny objects, and especially in machine vision where the goal is not an artistic effect, but rather to render a workpiece with as few artifacts as possible.

It would be hard to imagine an object that hasn't been viewed or photographed using polarized light. Certainly polarized light finds numerous uses even when imaging the eye, as for example for detecting drowsy vehicle drivers. Another example related to surgery can be found in patent publication US 2007/01436634A1, by LeBlanc et. al. It discloses relocating the usual off-axis eye illuminator to a more convenient on-axis position, and removing the specular reflections that would otherwise result by using polarized light.

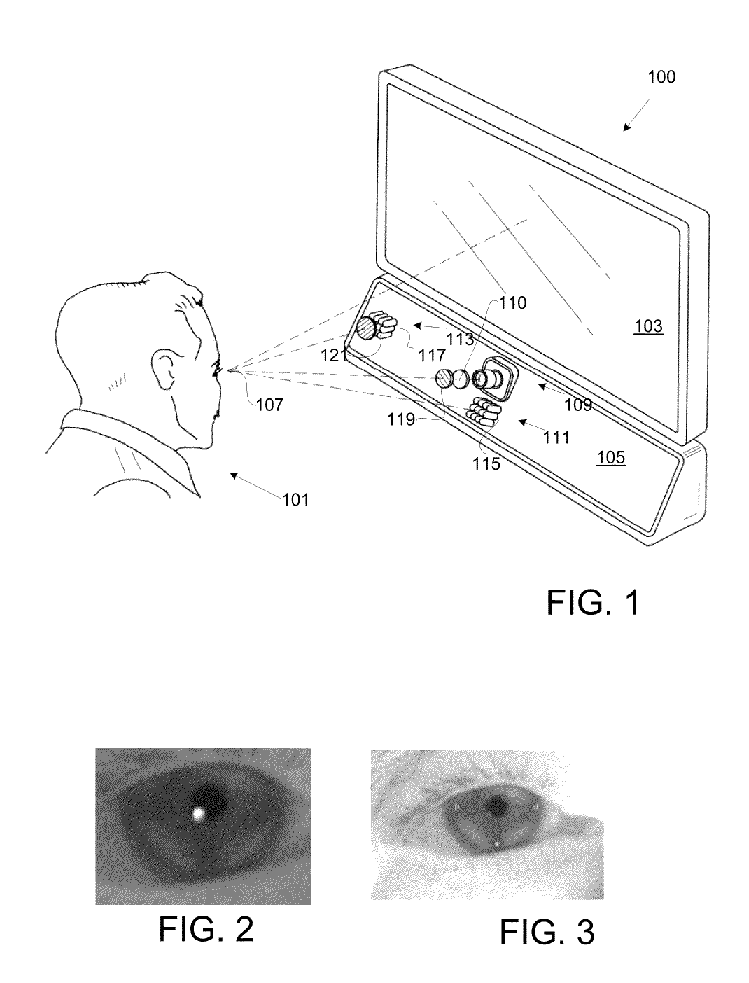

As shown in FIG. 1, a human viewer 101 is interacting with a computer system 100 including a display 103 and a gaze-tracking system 105 for tracking the motion of viewer eye 107. Gaze-tracking system 105 includes a camera 109, a “glint” illuminator 111, and a “pupil” illuminator 113. Illuminators 111 and 113 include respective LED arrays 115 and 117, which both emit infra-red light invisible to eye 107 but detectable by camera 109. Illuminators 111 and 113 are sufficiently bright that they can overcome ambient light. Camera 109 includes a near infra-red (NIR) filter 110 to block visible light. LED arrays 115 and 117 illuminate the eye from below with NIR light. In alternative embodiments, visible light is used to illuminate.

Light that reaches camera 109 first passes through a polarizing filter 119. Pupil illuminator 113 includes a polarizing filter 121 mounted thereon. In an alternative embodiment, the incoming polarizer is mounted to the camera. Polarizing filters 119 and 121 are cross polarized so that reflections of light from array 113 off of eye 107 are attenuated relative to light scattered by eye 107. In the illustrated embodiment, polarizing filters 119 and 121 are linear polarizers. Alternative embodiments variously use beam splitters and circular polarizers.

Since a glint is a reflection, while scattered light is used to image the iris and pupil, the polarizers have the effect of removing glint from this pupil image, making it easier to determine pupil position precisely. This effect can be recognized by comparing the glint image region of FIG. 2 with the pupil image region of FIG. 3. In one mode of operation, gaze tracking is performed on both eyes.

Camera 109 images an approximately 10″ wide swath of the face to a resolution of 1000 pixels. This means individual pixels are only 0.010″ apart, and thus a 0.10″ pupil will only image 10 pixels wide. Further the glint will only move about 0.1″ across the eye, or 10 pixels, as one looks from side to side on a 10″ wide screen viewed from 24″. Accordingly, glint and pupil positions are measured with a precision of about 0.1 pixel to allow a resolution of about 100 points across the screen, which, even tolerating some jitter, is sufficient for applications of gaze tracking such as cursor control.

One advantage of obtaining separate glint and pupil images is that polarization can be used to attenuate the glint in one (glint) image and not the other (pupil image). Another advantage is that the overall brightness of each image can be adjusted for optimal detection of the intended subject. For example, the overall brightness of the pupil image of FIG. 3 can be at least 50% greater than that of the glint image of FIG. 2; in this case, a dark pupil contrasts more strongly with the bright overall image, while the bright glint contrasts more strongly with the darker overall image. Although it depicts a pupil as well as a glint, the glint image of FIG. 2 is used to locate the glint and not the pupil.

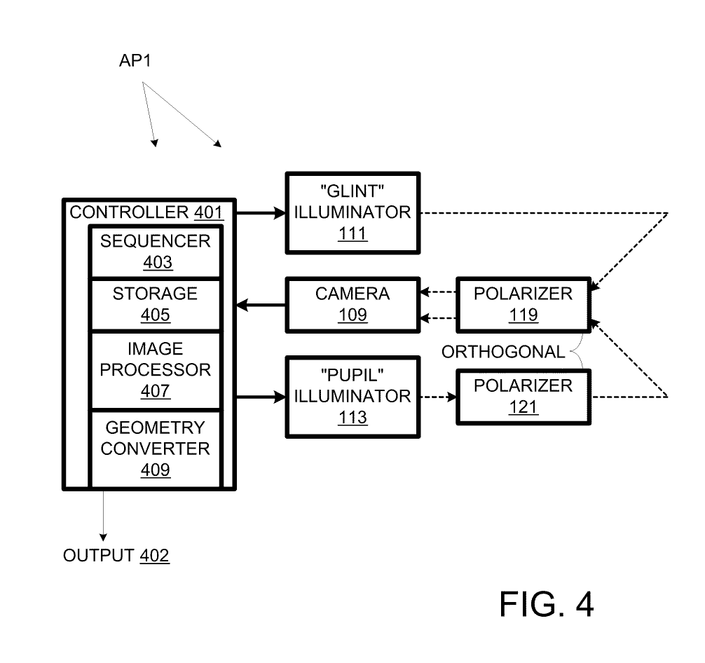

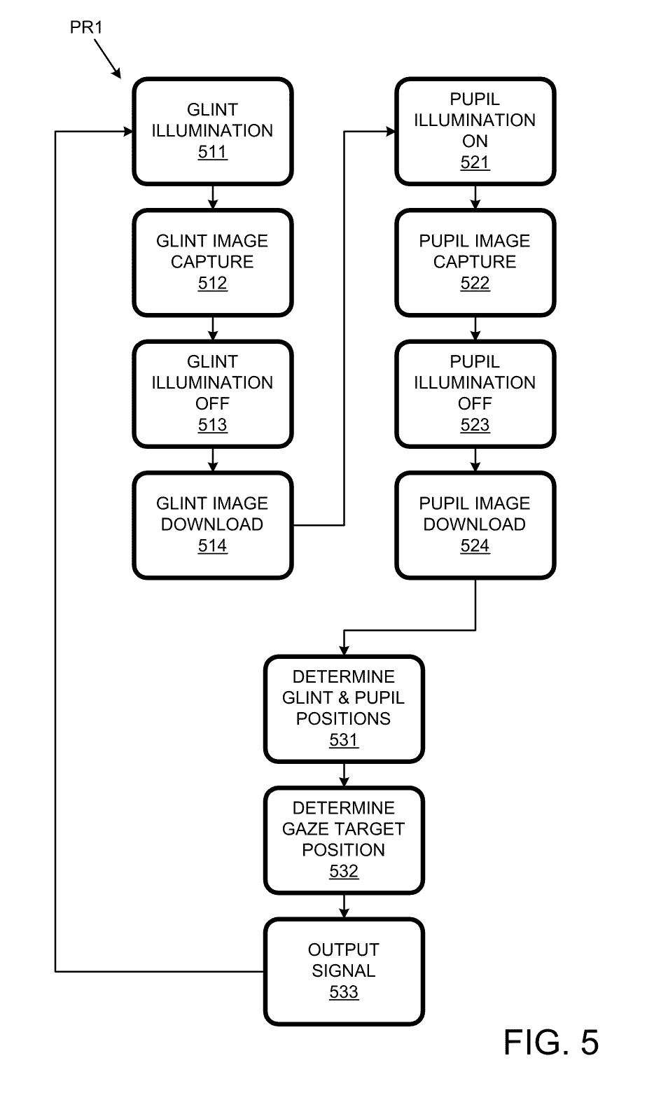

As shown in FIG. 4, gaze-tracking system AP1 includes a controller 401, camera 109, glint illuminator 111, pupil illuminator 113, and polarizers 119 and 121. Controller 401 includes a sequencer 403, storage media 405, an image processor 407, and a geometry converter 409. Storage media 405 is used for storing glint and pupil images, as well as for storing the results of image comparisons and analysis. Image processor 407 compares and analyzes glint and pupil images to determine glint and pupil centroids, which can be treated respectively as the (unextrapolated) glint and pupil positions.

As in the prior art, the center of the pupil is found by modeling it as a circle, and finding as many points on its perimeter as possible to be able to determine its center with a high degree of accuracy. A serious problem in the prior art is that the glint takes a huge bite out of the perimeter of the pupil, as depicted in FIG. 2. So, with some tens of percent of the dividing line between the pupil and the iris obscured, there is less information available to calculate the center of the pupil. The matter is only made worse by the often obscuring of the upper edge of the pupil by a drooping eyelid. Hence, the present invention addresses this problem by providing improved images revealing more of the pupil perimeter as the raw data for locating the pupil.

Geometry converter 409 converts these positions into a gaze target position, yielding an output 402, e.g., a control signal such as a cursor control signal (as might otherwise be generated by a mouse or trackball).

Sequencer 403 sequences process PR1, flow charted in FIG. 5, which is used to generate and analyze the glint and pupil images to determine gaze target position. At process segment 511, sequencer 403 turns on glint illuminator 111 so as to illuminate eye 107. In practice, head movement must be allowed so illuminator 111 can be situated to illuminate an area much larger than one eye. While glint illuminator 111 is on, e.g., for a few tens of milliseconds (ms), sequencer 403 commands camera 109 to capture an image at process segment 512. The result can be a glint image such as that shown in FIG. 2. At process segment 513, glint illuminator 111 is turned off to save power and so as not to interfere with obtaining a pupil image. At process segment 514, the captured glint image is downloaded to storage media 405.

The brightness values in the glint image (and the pupil image) can range from zero to 255. In the glint image, the glint itself is or will approach 255. A typical threshold of 225 can be used to detect the glint in the glint image. In the prior art, because a single image it taken, the exposure must be a compromise between being bright enough to reveal the pupil and iris, yet dim enough to reveal the glint. However, the current invention takes separate images of the pupil and glint, allowing the exposure of each image to be optimized separately.

In process segments 521-524, sequencer 403 repeats segments 511-514 but to obtain a pupil image instead of a glint image. At process segment 521, sequencer turns on pupil illuminator 113. The exposure will be greater than for the glint image to obtain a brighter image despite the attenuating effects of the polarizers; for example, the pupil exposure can be at least 50% and, in practice, 300% of the glint exposure. This higher exposure more than compensates for the loss of light due to the effect of camera polarizer 121. Alternatively, the pupil illuminator 113 can be made brighter than the glint illuminator 111. The bright exposure for the pupil image also lifts the exposure level out of the noise floor of the camera and increases the detectability of features such as the dividing line between a dark iris and a dark pupil, or between a bright iris and a bright pupil. In addition, the pupil illumination is polarized due to the presence of polarizing filter 121 to attenuate glint, e.g., by three or four orders of magnitude.

At process segment 522, sequencer 403 commands camera 109 to capture an image, in this case a pupil image such as that represented in FIG. 3. Any glint reflections are attenuated due to the cooperative action of polarizing filters 119 and 121, thus enhancing the detectability of the pupil. At process segment 523, pupil illumination is turned off. At process segment 524, the pupil image is downloaded to storage media 405. In alternative embodiments, the order of the process segments can be varied; for example, illuminators can be turned off after or during a download rather than before the downloading begins.

At process segment 531, the glint and pupil images are analyzed to determine glint and pupil positions. For example, centroids for the glint in the current glint image for the pupil in the current pupil image are obtained. The glint and pupil positions (coordinates) can be compared (subtracted) to subsequently determine a gaze target position at process segment 532. In effect, the images are superimposed and treated as a single image so that the position of the pupil is determined relative to the position of the glint as in the prior art.

The process for finding the glint starts with searching for the brightest pixels. To eliminate bright pixels from glints off of glasses frames, a check can be made for a proximal pupil. Next, a correlation is performed on the glint by taking an expected image of the glint and translating it vertically and horizontally for a best fit.

The pupil position can be determined and expressed in a number of ways. For example, the position of the pupil can be expressed in terms of the position of its center. The center can be determined, for example, by locating the boundary between the pupil and the iris and then determining the center of that boundary. In an alternative embodiment, the perimeter of the iris (the boundary between the iris and the sclera) is used to determine the pupil position.

To compensate for movement between the times the glint and pupil images are obtained, one or both of the glint and pupil positions can be extrapolated so that the two positions correspond to the same instant in time. To this end, one or more previously obtained glint and/or pupil images can be used. In an example, the cycle time for process PR1 is 40 ms and the pupil image is captured 10 ms after the corresponding glint image. Comparison of the glint positions indicates a head velocity of 4 pixels in 40 millseconds. This indicates a movement of 1 pixel in 10 ms. Thus, at the time the pupil image is captured, the glint position should be one pixel further in the direction of movement than it is in the actual current glint image. This extrapolated glint position that is compared to the unextrapolated pupil position obtained from the pupil image.

At process segment 532, the calculations involved in determining a gaze target position take into account the distance of the subject from the camera. This can be determined conventionally, e.g., using two cameras or measuring changes in the distance between the eyes. In other cases, an additional LED array can be used to make a second glint; in that case the distance between the glints can be measured.

A number of factors are taken into account to determine, from the glint and pupil positions in their respective images, where (e.g., on a computer screen) a person is actually gazing. These factors include the starting position of the user's eye relative to the screen and the camera, the instantaneous position of the user's eye with respect to the same, the curvature of the cornea, the aberrations of the camera lens, the cosine relationship between gaze angle and a point on the screen, and the geometry of the screen. These mathematical corrections are performed in software, and are well known in the art. Often several corrections can be lumped together and accommodated by having the user first “calibrate” the system. This involves having the software position a target on several predetermined points on the screen, and then for each, recording where the user is gazing. Jitter is often removed by averaging or otherwise filtering several gaze target positions before presenting them.

At process segment 533, the determined gaze target position can be used in generating output signal 402, e.g., a virtual mouse command, which can be used to control a cursor or for other purposes. Sequencer 403 then iterates process PR1, returning to process segment 511. Note that if the objective is a control signal rather than the gaze direction itself that is of interest, the gaze target position need not be explicitly determined. It also may not be necessary to determine the gaze target explicitly in an application that involves tracking head motion or determining the direction of eye movement. For example, in some applications, the direction of eye movement can represent a response (right=yes, left=no) or command.

The invention provides for many variations upon and modifications to the embodiments described above. In an embodiment, the pupil illuminator includes more than one array of LEDs, e.g., more than one pupil illuminator is used. In another embodiment, the pupil illuminator and/or glint illuminator includes a circular array of LEDs around the camera lens. For example, the pupil illuminator can include a circular array around the lens and an array of LEDs away from the lens. The circular array can be used when a “red pupil” (aka “bright pupil”) mode is selected, while the remote array can be used when “black pupil” (aka “dark pupil”) mode is selected. Also, various arrangements (positions and angles) of illuminators can be used to minimize shadows (e.g., by providing more diffuse lighting) and to reduce the effect of head position on illumination. Illuminators can be spread horizontally to correspond to a landscape orientation of the camera. Depending on the embodiment, the camera and illuminators can be head mounted (including helmet or eyeglasses) or “remote”, i.e., not attached to the user.

To reduce or eliminate the need for motion compensation, the latency between the times of the glint and pupil images can be minimized. In an alternative embodiment, the camera permits two images to be captured without downloading in between. In another embodiment, glint and pupil images are captured by separate cameras to minimize the delay. In some embodiments, polarization is achieved using polarizing beam splitters.

In this specification, related art is discussed below for expository purposes. Related art labeled “prior art” is admitted prior art; related art not labeled “prior art” is not admitted prior art. The embodiments described above, variations thereupon, and modifications thereto are within the subject matter defined by the following claims.

What is claimed is:

1. A process comprising:
illuminating at least one eye to produce a glint on said eye;
obtaining a glint image of an eye showing said glint on said eye;
illuminating said eye using polarized light;
obtaining a pupil image of said eye using a polarizer to attenuate reflected polarized light; and
determining at least one glint position at least in part from said glint image and at least one pupil position at least in part from said pupil image.

2. A process as recited in claim 1 further comprising determining a gaze target position of said eye at least in part by comparing said glint position with said pupil position.

3. A process as recited in claim 1 further comprising determining a position and orientation of said eye at least in part by comparing said glint position with said pupil position.

4. A process as recited in claim 1 further comprising determining a gaze direction of said eye at least in part by comparing said glint position with said pupil position.

5. A process as recited in claim 1 wherein the overall brightness of said pupil image is different from the overall brightness of said glint image.

6. A process as recited in claim 1 wherein the overall brightness of said pupil image is greater than the overall brightness of said glint image.

7. A process as recited in claim 1 wherein the overall brightness of said pupil image is at least 50% greater than the overall brightness of said glint image.

8. A process as recited in claim 1 wherein at least one of said pupil position and said glint position is an extrapolated position.

9. A process as recited in claim 8 wherein at least one previously obtained glint or pupil image is used in obtaining said extrapolated position.

10. A system comprising:
one or more cameras for obtaining glint and pupil images;
a glint illuminator for illuminating at least one eye to produce at least one glint that is represented in said glint image;
a pupil illuminator for illuminating said at least one eye so that at least one pupil is represented in said pupil image;
polarizers in an optical path between said pupil illuminator and said camera, said polarizers cooperating to attenuate light reflected by said at least one eye relative to light scattered by said at least one eye; and
a controller for causing said glint and pupil images to be obtained within one second of each other and for analyzing said images so as to compare at least one glint position with at least one pupil position, said at least one glint position being determined at least in part from said glint image, said at least one pupil position being determined from said at least one pupil image.

11. A system as recited in claim 10 wherein said controller determines a gaze target position at least in part as a function of said glint and pupil images.

12. A system as recited in claim 10 wherein said controller controls the exposures for said glint and pupil images so that the overall brightness of said pupil image is at least 50% greater than the overall brightness of said glint image.

13. A system as recited in claim 10 wherein at least one of said polarizers is a polarizing beam splitter.

14. A process as recited in claim 10 wherein said polarizers are linear polarizers.

15. A process as recited in claim 10 wherein said polarizers are circular polarizers.

16. A system as recited in claim 10 wherein said illuminators provide infrared light.

17. A system as recited in claim 10 wherein said controller provides for extrapolating at least one of said glint and pupil positions to obtain glint and pupil positions corresponding to the same instant in time.

18. A system as recited in claim 17 wherein said controller uses an image obtained before said glint and said pupil images were obtained when extrapolating said at least one of said glint and pupil positions.
